(19) United States
(12) Patent Application Publication
(10) Pub. No.: US 2014/0005055 A1
(43) Pub. Date: Jan. 2, 2014

(54) METHODS FOR IMPROVING GENOME ASSEMBLIES

(71) Applicant: Los Alamos National Security, LLC, Los Alamos, NM (US)

(72) Inventors: Xiaojing Zhang, Los Alamos, NM (US); Karen Walston Davenport, Los Alamos, NM (US); Lance Duane Green, Jemez Springs, NM (US); Shunsheng Han, Los Alamos, NM (US)

(73) Assignee: Los Alamos National Security, LLC

(21) Appl. No.: 13/931,342

(22) Filed: Jun. 28, 2013

Related U.S. Application Data
(60) Provisional application No. 61/666,634, filed on Jun. 29, 2012.

(51) Int. Cl. C12Q 1/68

(52) U.S. Cl. CPC C12Q 1/6874
USPC 506/2

(57) ABSTRACT

Advances in sequencing technologies have dramatically reduced costs in producing high quality draft genomes. There are still many contigs and possible misassembled regions in those draft genomes. Described herein are methods for improving the quality of sequencing techniques, and particularly methods for overcoming the loading bias inherent in, for instance, the PacBio sequencing process. Compared to Sanger sequencing technology, the herein described method is not only cost-effective but also can close gaps greater than 2.5 Kb in a single round of reactions. It can also sequence through high GC regions and difficult secondary structures such as hairpin loops.

### CROSS-REFERENCE TO RELATED APPLICATION(S)

This application claims the benefit of the earlier filing date of U.S. Provisional application No. 61/666,634, filed Jun. 29, 2012; the entire content of that prior application is incorporated herein in its entirety.

### STATEMENT OF GOVERNMENT SUPPORT

This invention was made with government support under Contract No. DEAC52-06NA25396 awarded by the U.S. Department of Energy. The government has certain rights in the invention.

### FIELD

This disclosure is in the field of nucleic acid sequencing, including methods and systems for improving the output of sequencing reactions such as single-molecule sequencing (so called third generation sequencing).

### BACKGROUND

Advances in sequencing technologies have dramatically reduced costs in producing high quality draft genomes. There are still many contigs and possible misassembled regions in those draft genomes. Improving the quality of these genomes requires an efficient and economical means to close gaps and resequence some regions in the genomes.

Second-generation sequencing technologies produce more and more draft genomes at an ever faster speed and lower cost. However, finished high quality genomes are still preferably used by researchers (Chain et al., Science 326:236-237, 2009). Closing gaps in a draft genome is necessary to improve the quality of the genome. Picking primers at gap regions for PCR and assembling the resulting PCR sequences into the genome can reduce numbers of both contigs and scaffolds. Since the advancement of much less expensive sequencing technologies, Sanger sequencing (Sanger et al., Nature 265:687-695, 1977; Sanger et al., Proc Natl Acad Sci USA, 70, 1209-1213, 1973) of individual PCR products spanning targeted regions becomes a more expensive method compared to the cost of the draft itself. Pooling dozens of PCR products of various sizes and sequencing them as one library with single molecular sequencing technology from PacBio is a much more economical option (McCarthy, Chem Biol. 17:675-676, 2010; Schadt et al., Hum Mol Genet. 19(R2):R227-240, 2010).

However, there is a loading bias against large DNA fragments in the PacBio sequencing process. The PacBio technique uses single molecule sequencing done in wells (e.g., zero-mode waveguides, ZMWs) on a chip, which is called a Single Molecule Real Time (SMRT) cell. Smaller PCR products will load into the PacBio wells with a much greater efficiency than larger PCR products. When PCR products ranging from 500 bp to 5 Kb are pooled and sequenced together using PacBio, the smaller products have a substantially higher coverage than the larger products resulting in poor quality or incomplete sequences for the larger PCR products.

### SUMMARY

Provided herein in a first embodiment is a method of sequencing a pool of at least two amplicons having different lengths, the method involving mixing an amount of a first amplicon with an amount of a second amplicon, wherein the amounts of the first and second amplicons are selected so there is a molar excess of the longer of the two amplicons in the resultant pooled amplicons; and subjecting the pooled amplicons to a nucleic acid sequencing reaction.

Another provided embodiment is an improved method for single-molecule real-time (SMRT) sequencing a pool of amplicons having different lengths, wherein the improvement comprises adjusting the amount of at least two of the amplicons included in the pool using the following formula: Volume=[PCR size (Kb)]²×[10 ng/PCR concentration (ng/μl)].

Also provided herein is a method for gap-filling sequencing of at least one amplicon, which method involves subjecting the amplicon to serial sequencing to produce a series of subreads of the same amplicon template; selecting a subset of the subreads based on the accuracy of the sequence of a portion of the amplicon; and using the sequences of the subset of subreads to assemble a consensus sequence for the amplicon. Optionally, the serial sequencing comprises single-molecule real-time (SMRT) sequencing.

The foregoing and other features and advantages will become more apparent from the following detailed description of several embodiments, which proceeds with reference to the accompanying figure(s).

### BRIEF DESCRIPTION OF THE FIGURE

FIG. 1 is a graph illustrating changes in coverage of PCR products by PacBio subreads as the relative molar amount of pooled PCR products is changed. Three groups of 18 PCR products with sizes ranging from 500 bp to 5 Kb were pooled in three PacBio libraries according to mass or molar amount and sequenced. Group 1 (left bar in each set) with Constant (equal) Mass for all PCR resulted in much higher coverage for the smaller PCR products while the longer PCR products were barely covered. Group 2 (middle bar in each set) with Constant (equal) Molar amount had an improvement in coverage for the larger products, but still less than the coverage for the smaller products. Group 3 (right bar in each group) with adjusted Molar amount by PCR Length shows dramatic improvement in the coverage for the larger products.

### DETAILED DESCRIPTION

### I. Abbreviations

CCS Circular Consensus Sequence

cDNA complementary DNA

FRET Förster (or Fluorescence) Resonance Energy Transfer

LRET Luminescence Resonance Energy Transfer

PacBio Pacific Biosciences

PEG Polyethylene Glycol

PCR Polymerase Chain Reaction

SMRT Single Molecule Real Time (sequencing)

ZMW Zero-Mode Waveguide

### II. Terms

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

In order to facilitate review of the various embodiments of the invention, the following explanations of specific terms are provided:

Active site: The catalytic site of an enzyme or antibody, such as the region of a polymerase where the chemical reaction (polymerization) occurs. The active site includes one or more residues or atoms in a spatial arrangement that permits interaction with the substrate(s) to effect the reaction of the latter.

Amplification: An increase in the amount of (number of copies of) nucleic acid molecules (DNA or RNA-to-DNA), wherein the sequence of the increased molecules is the same as or complementary to the nucleic acid template. An example of amplification is the polymerase chain reaction (PCR), in which a sample containing nucleic acid template is contacted with a pair of oligonucleotide primers (one of which binds upstream to the target sequence, the other of which downstream and on the opposing strand), under conditions that allow for the hybridization (annealing) of the primers to nucleic acid template in the sample. The primers are extended under suitable conditions (though nucleic acid polymerization). If additional copies of the nucleic acid are desired, the first copy is dissociated from the template, and additional copies of the primers (usually contained in the same reaction mixture) are annealed to the template and first copy, extended, and dissociated; this process is repeated to amplify the desired number of copies of the nucleic acid.

The products of amplification may be characterized by myriad techniques, including for instance electrophoresis, restriction endonuclease cleavage patterns, hybridization, nucleic acid sequencing, and other techniques known in the art.

Other examples of amplification techniques include reverse-transcription PCR (RT-PCR); strand displacement amplification (see U.S. Pat. No. 5,744,311); transcription-free isothermal amplification (see U.S. Pat. No. 6,033,881); repair chain reaction amplification (see WO 90/01069); ligase chain reaction amplification (see EP-A-320 308); gap filling ligase chain reaction amplification (see U.S. Pat. No. 5,427,930); coupled ligase detection and PCR (see U.S. Pat. No. 6,027,889); and NASBA™ RNA transcription-free amplification (see U.S. Pat. No. 6,025,134).

Further examples of amplification techniques include methods of whole genome amplification, such as degenerate oligonucleotide primed PCR (DOP-PCR), primer extension pre-amplification PCR (PEP-PCR), ligation-mediated PCR, and multiple displacement amplification (MDA).

Binding: An association between two or more molecules, such as the formation of a complex. Generally, the stronger the binding of the molecules in a complex, the slower their rate of dissociation. Specific binding refers to a preferential binding between an agent and a target.

Particular examples of specific binding include, but are not limited to, hybridization of one nucleic acid molecule to a complementary nucleic acid molecule, and the association of a protein (such as a polymerase) with a target protein or nucleic acid molecule.

In a particular example, a protein is known to bind to a nucleic acid molecule if a sufficient amount of the protein forms non-covalent chemical bonds to the nucleic acid molecule, for example a sufficient amount to permit detection of that binding.

In one example, an oligonucleotide molecule (such as an primer) is observed to bind to a target nucleic acid molecule if a sufficient amount of the oligonucleotide molecule forms base pairs or is hybridized to its target nucleic acid molecule to permit detection of that binding. The binding between an oligonucleotide and its target nucleic acid molecule is frequently characterized by the temperature (Tₘ) at which 50% of the oligonucleotide is melted from its target. A higher (Tₘ) means a stronger or more stable complex relative to a complex with a lower (Tₘ).

Chemical moiety: A portion or functional group of a molecule. Examples include an agent, such as a nucleotide, that is capable of reversibly binding to the template strand of a target nucleic acid molecule by specifically binding with a complementary nucleotide in the target nucleic acid molecule. In particular examples, the chemical moiety is attached to a probe via a molecular linker, and does not detach from the linker when the chemical moiety specifically binds to a complementary nucleotide on the target nucleic acid molecule.

Particular examples of chemical moieties include, but are not limited to, nucleotide analogs that can be incorporated into a growing complementary nucleic acid strand, such as a labeled nucleotide analog.

cDNA (complementary DNA): A piece of DNA lacking internal, non-coding segments (introns) and regulatory sequences that determine transcription. cDNA is synthesized in the laboratory by reverse transcription from messenger RNA extracted from cells.

Complementary: A double-stranded DNA or RNA strand consists of two complementary strands of base pairs. Since there is one complementary base for each base found in DNA/RNA (such as A/T, and C/G), the complementary strand for any single strand can be determined.

De Novo Circular Consensus Sequence (CCS) Read: The consensus sequence produced by a PacBio sequencing system from the alignment of subreads taken from a single ZMW.

Detect: To determine if an agent is present or absent. In some examples this can further include quantification. For example, use of the disclosed probes in particular examples permits detection of a chemical moiety, for example as the chemical moiety binds to a complementary nucleotide in the target nucleic acid molecule without being detached from the linker.

Detection can be in bulk, so that a macroscopic number of molecules (such as at least 10²³ molecules) can be observed simultaneously. Detection can also include identification of signals from single molecules using microscopy and such techniques as total internal reflection to reduce background noise. The spectra of individual molecules can be obtained by these techniques (Ha et al., Proc. Natl. Acad. Sci. USA. 93:6264-6268, 1996).

Electromagnetic radiation: A series of electromagnetic waves that are propagated by simultaneous periodic variations of electric and magnetic field intensity, and that includes radio waves, infrared, visible light, ultraviolet light, X-rays and gamma rays. In particular examples, electromagnetic radiation is emitted by a laser, which can possess properties of mono-chromaticity, directionality, coherence, polarization, and intensity. Lasers are capable of emitting light at a particular wavelength (or across a relatively narrow range of wavelengths), such that energy from the laser can excite a donor but not an acceptor fluorophore.

Emission signal: The light of a particular wavelength generated from a fluorophore after the fluorophore absorbs light at its excitation wavelengths.

Emission or emission signal: The light of a particular wavelength generated from a source. In particular examples, an emission signal is emitted from a fluorophore after the fluorophore absorbs light at its excitation wavelength(s).

Emission spectrum: The energy spectrum which results after a fluorophore is excited by a specific wavelength of light. Each fluorophore has a characteristic emission spectrum. In one example, individual fluorophores (or unique combinations of fluorophores) are associated with a nucleotide analog and the emission spectra from the fluorophores provide a means for distinguishing between the different nucleotide analogs.

Electrophoresis: Electrophoresis refers to the migration of charged solutes or particles in a liquid medium under the influence of an electric field. Electrophoretic separations are widely used for analysis of macromolecules. Of particular importance is the identification of proteins and nucleic acid sequences. Such separations can be based on differences in size and/or charge. Nucleotide sequences have a uniform charge and are therefore separated based on differences in size. Electrophoresis can be performed in an unsupported liquid medium (for example, capillary electrophoresis), but more commonly the liquid medium travels through a solid supporting medium. The most widely used supporting media are gels, for example, polyacrylamide and agarose gels.

Sieving gels (for example, agarose) impede the flow of molecules. The pore size of the gel determines the size of a molecule that can flow freely through the gel. The amount of time to travel through the gel increases as the size of the molecule increases. As a result, small molecules travel through the gel more quickly than large molecules and thus progress further from the sample application area than larger molecules, in a given time period. Such gels are used for size-based separations of nucleotide sequences.

Fragments of linear DNA migrate through agarose gels with a mobility that is inversely proportional to the log₁₀ of their molecular weight. By using gels with different concentrations of agarose, different sizes of DNA fragments can be resolved. Higher concentrations of agarose facilitate separation of small DNAs, while low agarose concentrations allow resolution of larger DNAs.

Excitation or excitation signal: The light of a particular wavelength necessary and/or sufficient to excite an electron transition to a higher energy level. In particular examples, an excitation signal is the light of a particular wavelength necessary and/or sufficient to excite a fluorophore to a state such that the fluorophore will emit a different (such as a longer) wavelength of light than the wavelength of light from the excitation signal.

Fluorophore: A chemical compound, which when excited by exposure to a particular stimulus such as a defined wavelength of light, emits light (fluoresces), for example at a different wavelength.

Fluorophores are part of the larger class of luminescent compounds. Luminescent compounds include chemiluminescent molecules, which do not require a particular wavelength of light to luminesce, but rather use a chemical source of energy. Therefore, the use of chemiluminescent molecules eliminates the need for an external source of electromagnetic radiation, such as a laser. Examples of chemiluminescent molecules include, but are not limited to, aequorin (Tsien, 1998, Ann. Rev. Biochem. 67:509).

Examples of particular fluorophores are provided in U.S. Pat. No. 5,866,366 to Nazarenko et al., such as 4-acetamido-4′-isothiocyanatostilbene-2,2′ disulfonic acid, acridine and derivatives such as acridine and acridine isothiocyanate, 5-(2′-aminoethyl)aminonaphthalene-1-sulfonic acid (EDANS), 4-amino-N-[3-vinylsulfonyl)phenyl]naphthalimide-3,5 disulfonate (Lucifer Yellow VS), N-(4-anilino-1-naphthyl)maleimide, anthranilamide, Brilliant Yellow, coumarin and derivatives such as coumarin, 7-amino-4-methylcoumarin (AMC, Coumarin 120), 7-amino-4-trifluoromethylcouluarin (Coumaran 151); cyanosine; 4′,6-diaminidino-2-phenylindole (DAPI); 5′,5″-dibromopyrogallol-sulfonephthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansyl chloride); 4-dimethylaminophenylazophenyl-4′-isothiocyanate (DABITC); eosin and derivatives such as eosin and eosin isothiocyanate; erythrosin and derivatives such as erythrosin B and erythrosin isothiocyanate; ethidium; fluorescein and derivatives such as 5-carboxyfluorescein (FAM), 5-(4,6-dichlorotriazin-2-yl)aminofluorescein (DTAF), 2′7′-dimethoxy-4′5′-dichloro-6-carboxyfluorescein (JOE), fluorescein, fluorescein isothiocyanate (FITC), and QFITC (XRITC); fluorescamine; IR144; IR1446; Malachite Green isothiocyanate; 4-methylumbelliferone; ortho cresolphthalein; nitrotyro sine; pararosaniline; Phenol Red; B-phycoerythrin; o-phthaldialdehyde; pyrene and derivatives such as pyrene, pyrene butyrate and succinimidyl 1-pyrene butyrate; Reactive Red 4 (Cibacron® Brilliant Red 3B-A); rhodamine and derivatives such as 6-carboxy-X-rhodamine (ROX), 6-carboxyrhodamine (R6G), lissamine rhodamine B sulfonyl chloride, rhodamine (Rhod), rhodamine B, rhodamine 123, rhodamine X isothiocyanate, sulforhodamine B, sulforhodamine 101 and sulfonyl chloride derivative of sulforhodamine 101 (Texas Red); N,N,N′,N′-tetramethyl-6-carboxyrhodamine (TAMRA); tetramethyl rhodamine; tetramethyl rhodamine isothiocyanate (TRITC); riboflavin; rosolic acid and terbium chelate derivatives.

Other suitable fluorophores include thiol-reactive europium chelates which emit at approximately 617 nm (Heyduk and Heyduk, Analyt. Biochem. 248:216-27, 1997; J. Biol. Chem. 274:3315-22, 1999), as well as GFP, Lissamine™, diethylaminocoumarin, fluorescein chlorotriazinyl, naphthofluorescein, 4,7-dichlororhodamine and xanthene (as described in U.S. Pat. No. 5,800,996 to Lee et al.) and derivatives thereof. In one example, the fluorophore is a large Stokes shift protein (see Kogure et al., Nat. Biotech. 24:577-81, 2006). Other fluorophores known to those skilled in the art can also be used, for example those available from Molecular Probes (Invitrogen, Eugene, Oreg.).

In particular examples, a fluorophore is used as a donor fluorophore or as an acceptor fluorophore. The fluorophores can be used as donor fluorophores or as acceptor fluorophores. Particularly useful fluorophores have the ability to be attached (for example to a polymerase, a molecular linker, or to a nucleotide analog) are stable against photobleaching, and have high quantum efficiency. In addition, the fluorophores associated with different sets of nucleotide analogs (such as those that correspond to A, T/U, G, and C) are advantageously selected to have distinguishable emission spectra, such that emission from one fluorophore (such as one associated with A) is distinguishable from the fluorophore associated with another nucleotide analog (such as one associated with T).

“Acceptor fluorophores” are fluorophores which absorb energy from a donor fluorophore, for example in the range of about 400 to 900 nm (such as in the range of about 500 to 800 nm). Acceptor fluorophores generally absorb light at a wavelength which is usually at least 10 nm higher (such as at least 20 nm higher) than the maximum absorbance wavelength of the donor fluorophore, and have a fluorescence emission maximum at a wavelength ranging from about 400 to 900 nm. Acceptor fluorophores have an excitation spectrum that overlaps with the emission of the donor fluorophore, such that energy emitted by the donor can excite the acceptor. Ideally, an acceptor fluorophore is capable of being attached to a nucleic acid molecule. In a particular example, an acceptor fluorophore is a dark quencher, such as Dabcyl, QSY7 (Molecular Probes), QSY33 (Molecular Probes), BLACK HOLE QUENCHERS™ (Biosearch Technologies; such as BHQ0, BHQ1, BHQ2, and BHQ3), ECLIPSE™ Dark Quencher (Epoch Biosciences), or IOWA BLACK™ (Integrated DNA Technologies). A quencher can reduce or quench the emission of a donor fluorophore. In such an example, instead of detecting an increase in emission signal from the acceptor fluorophore when in sufficient proximity to the donor fluorophore (or detecting a decrease in emission signal from the acceptor fluorophore when a significant distance from the donor fluorophore), an increase in the emission signal from the donor fluorophore can be detected when the quencher is a significant distance from the donor fluorophore (or a decrease in emission signal from the donor fluorophore when in sufficient proximity to the quencher acceptor fluorophore).

“Donor Fluorophores” are fluorophores or luminescent molecules capable of transferring energy to an acceptor fluorophore, thereby generating a detectable fluorescent signal from the acceptor. Donor fluorophores are generally compounds that absorb in the range of about 300 to 900 nm, for example about 350 to 800 nm. Donor fluorophores have a strong molar absorbance coefficient at the desired excitation wavelength, for example greater than about 10³ M⁻¹ cm⁻¹. A variety of compounds can be employed as donor fluorescent components, including fluorescein (and derivatives thereof), rhodamine (and derivatives thereof), GFP, phycoerythrin, BODIPY, DAPI (4′,6-diamidino-2-phenylindole), Indo-1, coumarin, dansyl, terbium (and derivatives thereof), and cyanine dyes. In particular examples, a donor fluorophore is a chemiluminescent molecule, such as aequorin.

Förster (or Fluorescence) resonance energy transfer (FRET): A process in which an excited fluorophore (the donor) transfers its excited state energy to a lower-energy light absorbing molecule (the acceptor). This energy transfer is non-radiative, and due primarily to a dipole-dipole interaction between the donor and acceptor fluorophores. This energy can be passed over a distance, for example a limited distance such as 10-100 Å. FRET efficiency drops off according to 1/(1+(R/R0)⁶) where R0 is the distance at which the FRET efficiency is 50%.

Genome: The total genetic constituents of an organism. In the case of eukaryotic organisms, the genome is contained in a haploid set of chromosomes of a cell. In the case of prokaryotic organisms, the genome is contained in a single chromosome, and in some cases one or more extra-chromosomal genetic elements, such as episomes (e.g., plasmids). A viral genome can take the form of one or more single or double stranded DNA or RNA molecules depending on the particular virus.

Hybridization: Oligonucleotides and their analogs hybridize by hydrogen bonding, which includes Watson-Crick, Hoogsteen or reversed Hoogsteen hydrogen bonding, between complementary bases. Generally, nucleic acid consists of nitrogenous bases that are either pyrimidines (cytosine (C), uracil (U), and thymine (T)) or purines (adenine (A) and guanine (G)). These nitrogenous bases form hydrogen bonds between a pyrimidine and a purine, and the bonding of the pyrimidine to the purine is referred to as “base pairing.” More specifically, A will hydrogen bond to T or U, and G will bond to C. “Complementary” refers to the base pairing that occurs between two distinct nucleic acid sequences or two distinct regions of the same nucleic acid sequence.

“Specifically hybridizable” and “specifically complementary” are terms that indicate a sufficient degree of complementarity such that stable and specific binding occurs between the oligonucleotide (or its analog) and the DNA or RNA target. The oligonucleotide or oligonucleotide analog need not be 100% complementary to its target sequence to be specifically hybridizable. An oligonucleotide or analog is specifically hybridizable when binding of the oligonucleotide or analog to the target DNA or RNA molecule interferes with the normal function of the target DNA or RNA, and there is a sufficient degree of complementarity to avoid non-specific binding of the oligonucleotide or analog to non-target sequences under conditions where specific binding is desired, for example under physiological conditions in the case of in vivo assays or systems. Such binding is referred to as specific hybridization.

Hybridization conditions resulting in particular degrees of stringency will vary depending upon the nature of the hybridization method of choice and the composition and length of the hybridizing nucleic acid sequences. Generally, the temperature of hybridization and the ionic strength (especially the Na⁺ and/or Mg⁺⁺ concentration) of the hybridization buffer will determine the stringency of hybridization, though wash times also influence stringency. Calculations regarding hybridization conditions required for attaining particular degrees of stringency are discussed by Sambrook et al. (ed.), Molecular Cloning: A Laboratory Manual, 2^{nd} ed., vol. 1-3, Cold Spring Harbor Laboratory Press, Cold Spring Harbor, N.Y., 1989, chapters 9 and 11; and Ausubel et al. Short Protocols in Molecular Biology, 4^{th} ed., John Wiley & Sons, Inc., 1999.

Hybridization conditions resulting in particular degrees of stringency will vary depending upon the nature of the hybridization method and the composition and length of the hybridizing nucleic acid sequences. Generally, the temperature of hybridization and the ionic strength (such as the Na⁺ concentration) of the hybridization buffer will determine the stringency of hybridization. Calculations regarding hybridization conditions for attaining particular degrees of stringency are discussed in Sambrook et al., (1989) Molecular Cloning, second edition, Cold Spring Harbor Laboratory, Plainview, N.Y. (chapters 9 and 11). The following is an exemplary set of hybridization conditions and is not limiting:

Very High Stringency (Detects Sequences that Share at Least 90% Identity)

Hybridization: 5×SSC at 65° C. for 16 hours

Wash twice: 2×SSC at room temperature (RT) for 15 minutes each

Wash twice: 0.5×SSC at 65° C. for 20 minutes each

High Stringency (Detects Sequences that Share at Least 80% Identity)

Hybridization: 5×-6×SSC at 65° C.-70° C. for 16-20 hours

Wash twice: 2×SSC at RT for 5-20 minutes each

Wash twice: 1×SSC at 55° C.-70° C. for 30 minutes each

Low Stringency (Detects Sequences that Share at Least 50% Identity)

Hybridization: 6×SSC at RT to 55° C. for 16-20 hours

Wash at least twice: 2×-3×SSC at RT to 55° C. for 20-30 minutes each.

20×SSC is 3.0 M NaCl/0.3 M trisodium citrate.

Isolated: An “isolated” or “purified” biological component (such as a nucleic acid, peptide, protein, protein complex, or particle) has been substantially separated, produced apart from, or purified away from other biological components in the cell of the organism in which the component naturally occurs, that is, other chromosomal and extra-chromosomal DNA and RNA, and proteins. Nucleic acids, peptides and proteins that have been “isolated” or “purified” thus include nucleic acids and proteins purified by standard purification methods. The term also embraces nucleic acids, peptides and proteins prepared by recombinant expression in a host cell, as well as chemically synthesized nucleic acids or proteins. The term “isolated” or “purified” does not require absolute purity; rather, it is intended as a relative term. Thus, for example, an isolated biological component is one in which the biological component is more enriched than the biological component is in its natural environment within a cell, or other production vessel. Preferably, a preparation is purified such that the biological component represents at least 50%, such as at least 70%, at least 90%, at least 95%, or greater, of the total biological component content of the preparation.

Label: A detectable compound or composition that is conjugated directly or indirectly to another molecule to facilitate detection of that molecule. Specific, non-limiting examples of labels include fluorescent tags, enzymatic linkages, and radioactive isotopes.

Linker: A structure that joins one molecule to another, such as attaches a probe of the present disclosure to a substrate, wherein one portion of the linker is operably linked to a substrate, and wherein another portion of the linker is operably linked to the probe.

One particular type of linker is a molecular linker, such as tethers, rods, or combinations thereof, which can attach a polymerizing agent to one or more chemical moieties (such as one or more nucleotide analogs) wherein one portion of the linker is operably linked to the polymerizing agent, and wherein another portion of the linker is operably linked to one or more chemical moieties.

Luminescence Resonance Energy Transfer (LRET): A process similar to FRET, in which the donor molecule is a luminescent molecule, or is excited by a luminescent molecule, instead of for example by a laser. Using LRET can decrease the background fluorescence. In particular examples, a chemiluminescent molecule can be used to excite a donor fluorophore (such as GFP), without the need for an external source of electromagnetic radiation. In other examples, the luminescent molecule is the donor, wherein the excited resonance of the luminescent molecule excites one or more acceptor fluorophores.

Examples of luminescent molecules that can be used include, but are not limited to, aequorin and luciferase. The bioluminescence from aequorin, which peaks at 470 nm, can be used to excite a donor GFP fluorophore (Tsien, Ann. Rev. Biochem. 67:509, 1998; Baubet et al., 2000, Proc. Natl. Acad. Sci. U.S.A., 97:7260-7265). GFP then excites an acceptor fluorophore disclosed herein. The bioluminescence from Photinus pyralis luciferase, which peaks at 555 nm, can excite an acceptor fluorophore disclosed herein. In some examples where luciferase is used, the dipole of the acceptor fluorophore is aligned with the polarization of the luciferase light. For example, a sphere, a dendrimer or a sheet could be made that has many molecules of luciferase inside or on the surface.

Modified nucleotide (modified nucleoside triphosphate): A modified nucleotide is a nucleotide that has been altered, for example a nucleotide to which a chemical moiety has been added, often one that gives an additional functionality to the modified nucleotide. Generally, the modification comprises a functional group or a leaving group, such as permits coupling of the nucleotide to a detectable molecule, e.g., a fluorophore or hapten. The term also includes nucleotides containing a modified base, a modified sugar moiety, and/or a modified phosphate backbone, for example as described in U.S. Pat. No. 5,866,336.

Examples of modified sugar moieties which may be used at any position on its structure to modify a nucleotide include, but are not limited to: arabinose, 2-fluoroarabinose, xylose, and hexose. A modified component of the phosphate backbone includes, but is not limited to, a phosphorothioate, a phosphorodithioate, a phosphoramidothioate, a phosphoramidate, a phosphordiamidate, a methylphosphonate, an alkyl phosphotriester, or a formacetal or analog thereof.

Multiple displacement amplification (MDA): A method of replication (or amplification) of DNA that utilizes the strand displacement activity of certain DNA polymerases. The method generally involves hybridization of primers, for example random primers, such as random hexamers, to a template nucleic acid sequence, and replication of the sequence. During replication, the elongating strands displace other strands from the template sequence (or from another replicated strand) by strand displacement replication. Strand displacement replication refers to DNA replication (polymerization) where a growing end of a replicated strand encounters and displaces another strand from the template strand or another replicated strand. See U.S. Pat. Nos. 6,124,120 and 6,977,148, for instance.

Multiplex (e.g., PCR): Amplification of multiple nucleic acid species in a single amplification reaction, such as a single real-time PCR reaction. By multiplexing, target nucleic acids (including an endogenous control, in some examples) can be amplified in single tube, plate, chip, lane of a flow cell, or other reaction vessel or system. Sample multiplexing is a useful technique when targeting specific genomic regions or working with smaller genomes. Pooling samples exponentially increases the number of samples analysed in a single run without drastically increasing cost or time. To prepare samples for multiplexing, a unique identifier tag (in some contexts referred to as a barcode), or index, is added to the sequences in each library. Sequences from that sample library can be distinguished from pooled sequences based on the presence of the unique identifier tag sequence.

Nucleic acid molecule: A polymeric form of nucleotides, which may include both sense and anti-sense strands of RNA, cDNA, genomic DNA, and synthetic forms and mixed polymers of the above. A nucleotide refers to a ribonucleotide, deoxynucleotide or a modified form of either type of nucleotide. The term “nucleic acid molecule” as used herein is synonymous with “nucleic acid” and “polynucleotide.” A nucleic acid molecule is usually at least 10 bases in length, unless otherwise specified. The term includes single- and double-stranded forms of DNA. A polynucleotide may include either or both naturally occurring and modified nucleotides linked together by naturally occurring and/or non-naturally occurring nucleotide linkages.

Nucleotide: A monomer that includes a base, such as a pyrimidine, purine, or synthetic analogs thereof, linked to a sugar and one or more phosphate groups. A nucleotide is one monomer in a polynucleotide. A nucleotide sequence refers to the sequence of bases in a polynucleotide.

The major nucleotides of DNA are deoxyadenosine 5′-triphosphate (dATP or A), deoxyguanosine 5′-triphosphate (dGTP or G), deoxycytidine 5′-triphosphate (dCTP or C) and deoxythymidine 5′-triphosphate (dTTP or T). The major nucleotides of RNA are adenosine 5′-triphosphate (ATP or A), guanosine 5′-triphosphate (GTP or G), cytidine 5′-triphosphate (CTP or C) and uridine 5′-triphosphate (UTP or U).

The choice of nucleotide precursors is dependent on the nucleic acid to be sequenced. If the template is a single-stranded DNA molecule, deoxyribonucleotide precursors (dNTPs) are used in the presence of a DNA-directed DNA polymerase. Alternatively, ribonucleotide precursors (NTPs) are used in the presence of a DNA-directed RNA polymerase. However, if the nucleic acid to be sequenced is RNA, then dNTPs and an RNA-directed DNA polymerase are used.

The nucleotides disclosed herein also include nucleotides containing modified bases, modified sugar moieties and modified phosphate backbones, for example as described in U.S. Pat. No. 5,866,336 to Nazarenko et al. (herein incorporated by reference). Such modifications however, can allow for incorporation of the nucleotide into a growing nucleic acid chain or for binding of the nucleotide to the complementary nucleic acid chain. Modifications described herein do not result in the termination of nucleic acid synthesis.

Nucleotides can be modified at any position on their structures. Examples include, but are not limited to, the modified nucleotides 5-fluorouracil, 5-bromouracil, 5-chlorouracil, 5-iodouracil, hypoxanthine, xanthine, acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N˜6-sopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid, 5-methyl-2-thiouracil, 3-(3-amino-3-N-2-carboxypropyl)uracil, and 2,6-diaminopurine.

Examples of modified sugar moieties which can be used to modify nucleotides at any position on their structures include, but are not limited to: arabinose, 2-fluoroarabinose, xylose, and hexose, or a modified component of the phosphate backbone, such as phosphorothioate, a phosphorodithioate, a phosphoramidothioate, a phosphoramidate, a phosphordiamidate, a methylphosphonate, an alkyl phosphotriester, or a formacetal or analog thereof.

Nucleotide analog: A nucleotide containing one or more modifications of the naturally occurring base, sugar, phosphate backbone, or combinations thereof. Such modifications can result in the inability of the nucleotide to be incorporated into a growing nucleic acid chain. A particular example includes a non-hydrolyzable nucleotide. Non-hydrolyzable nucleotides include mononucleotides and trinucleotides in which the oxygen between the alpha and beta phosphates has been replaced with nitrogen or carbon (Jena Bioscience). HIV-1 reverse transcriptase cannot hydrolyze dTTP with the oxygen between the alpha and beta phosphates replaced by nitrogen (Ma et al., J. Med. Chem., 35: 1938-41, 1992).

A “type” of nucleotide analog refers to one of a set of nucleotide analogs that share a common characteristic that is to be detected. For example, the sets of nucleotide analogs can be divided into four types: A, T, C and G analogs (for DNA) or A, U, C and G analogs (for RNA). In this example, each type of nucleotide analog can be associated with a unique tag, such as one or more acceptor fluorophores, so as to be distinguishable from the other nucleotide analogs in the set (for example by fluorescent spectroscopy or by other optical means).

An exemplary nucleotide analog that can be used in place of “C” is a G-clamp (Glen Research). G-clamp is a tricyclic Aminoethyl-Phenoxazine 2′-deoxyCytidine analogue (AP-dC). The G-clamp is available as a phosphoramidite and so can be synthesized into DNA structures.

Oligonucleotide: A nucleic acid molecule generally comprising a length of 300 bases or fewer. The term often refers to single-stranded deoxyribonucleotides, but it can refer as well to single- or double-stranded ribonucleotides, RNA:DNA hybrids and double-stranded DNAs, among others. The term “oligonucleotide” also includes oligonucleosides (that is, an oligonucleotide minus the phosphate) and any other organic base polymer.

In some examples, oligonucleotides are about 10 to about 90 bases in length, for example, 12, 13, 14, 15, 16, 17, 18, 19 or 20 bases in length. Other oligonucleotides are about 25, about 30, about 35, about 40, about 45, about 50, about 55, about 60 bases, about 65 bases, about 70 bases, about 75 bases or about 80 bases in length. Oligonucleotides may be single-stranded, for example, for use as probes or primers, or may be double-stranded, for example, for use in the construction of a mutant gene. Oligonucleotides can be either sense or anti-sense oligonucleotides. An oligonucleotide can be modified as discussed above in reference to nucleic acid molecules. Oligonucleotides can be obtained from existing nucleic acid sources (for example, genomic or cDNA), but can also be synthetic (for example, produced by laboratory or in vitro oligonucleotide synthesis).

Open Reading Frame (ORF): A series of nucleotide triplets (codons) coding for amino acids without any internal termination codons. These sequences are usually translatable into a peptide/polypeptide/protein/polyprotein.

It is recognized in the art that the following codons (shown for RNA) can be used interchangeably to code for each specific amino acid or termination: Alanine (Ala or A) GCU, GCG, GCA, or GCG; Arginine (Arg or R) CGU, CGC, CGA, CGG, AGA, or AGG; Asparagine (Asn or N) AAU or AAC; Aspartic Acid (Asp or D) GAU or GAC; Cysteine (Cys or C) UGU or UGC; Glutamic Acid (Glu or E) GAA or GAG; Glutamine (Gln or Q) CAA or CAG; Glycine (Gly or G) GGU, GGC, GGA, or GGG; Histidine (H is or H) CAU or CAC; Isoleucine (Ile or I) AUU, AUC, or AUA; Leucine (Leu or L) UUA, UUG, CUU, CUC, CUA, or CUG; Lysine (Lys or K) AAA or AAG; Methionine (Met or M) AUG; Phenylalanine (Phe or F) UUU or UUC; Proline (Pro or P) CCU, CCC, CCA, or CCG; Serine (Ser or S) UCU, UCC, UCA, UCG, AGU, or AGC; Termination codon UAA (ochre) or UAG (amber) or UGA (opal); Threonine (Thr or T) ACU, ACC, ACA, or ACG; Tyrosine (Tyr or Y) UAU or UAC; Tryptophan (Trp or W) UGG; and Valine (Val or V) GUU, GUC, GUA, or GUG. The corresponding codons for DNA have T substituted for U in each instance.

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence is the promoter affects the transcription or expression of the coding sequence. Generally, operably linked DNA sequences are contiguous and, where necessary to join two protein-coding regions, in the same reading frame. If introns are present, the operably linked DNA sequences may not be contiguous.

Phospholinked nucleotide: For each of the nucleotide bases, there are four corresponding fluorescent dye molecules that enable a detector to identify the base being incorporated by the DNA polymerase as it performs the DNA synthesis. The fluorescent dye molecule is attached to the phosphate chain of the nucleotide. When the nucleotide is incorporated by the DNA polymerase, the fluorescent dye is cleaved off with the phosphate chain as a part of a natural DNA synthesis process during which a phosphodiester bond is created to elongate the DNA chain. The cleaved fluorescent dye molecule then diffuses out of the detection volume so that the fluorescent signal is no longer detected.

Polyethylene glycol (PEG): A polymer of ethylene, H(OCH₂CH₂)ₙOH. Pegylation is the act of adding a PEG structure to another molecule, for example, a functional molecule such as a targeting or activatable moiety. PEG is soluble in water, methanol, benzene, dichloromethane and is insoluble in diethyl ether and hexane. Particular examples of PEG include, but are not limited to: 1-7 units of Spacer 18 (Integrated DNA Technologies, Coralville, Iowa), such as 3-5 units of Spacer 18, C3 Spacer phosphoramidite (such as 1-10 units), Spacer 9 (such as 1-10 units), PC (Photo-Cleavable) Spacer (such as 1-10 units), (all available from Integrated DNA Technologies). In other examples, lengths of PEG that can be used in the disclosed methods include, but are not limited to, 1 to 40 monomers of PEG. PEG can optionally be used in size exclusion embodiments, for instance attached to a polymerase or other molecule.

Probes and primers: A probe comprises an isolated nucleic acid molecule attached to a detectable label or other reporter molecule. Typical labels include radioactive isotopes, enzyme substrates, co-factors, ligands, chemiluminescent or fluorescent agents, haptens, and enzymes. Methods for labeling and guidance in the choice of labels appropriate for various purposes are discussed, for example, in Sambrook et al. (ed.), Molecular Cloning: A Laboratory Manual, 2^{nd} ed., vol. 1-3, Cold Spring Harbor Laboratory Press, Cold Spring Harbor, N.Y., 1989 and Ausubel et al. Short Protocols in Molecular Biology, 4^{th} ed., John Wiley & Sons, Inc., 1999.

Primers are short nucleic acid molecules, for instance DNA oligonucleotides 6 nucleotides or more in length, for example that hybridize to contiguous complementary nucleotides or a sequence to be amplified. Longer DNA oligonucleotides may be about 10, 12, 15, 20, 25, 30, or 50 nucleotides or more in length. Primers can be annealed to a complementary target DNA strand by nucleic acid hybridization to form a hybrid between the primer and the target DNA strand, and then the primer extended along the target DNA strand by a DNA polymerase enzyme. Primer pairs can be used for amplification of a nucleic acid sequence, for example, by the polymerase chain reaction (PCR) or other nucleic-acid amplification methods known in the art. Other examples of amplification include strand displacement amplification, as disclosed in U.S. Pat. No. 5,744,311; transcription-free isothermal amplification, as disclosed in U.S. Pat. No. 6,033,881; repair chain reaction amplification, as disclosed in WO 90/01069; ligase chain reaction amplification, as disclosed in EP-A-320 308; gap filling ligase chain reaction amplification, as disclosed in 5,427,930; and NASBA™ RNA transcription-free amplification, as disclosed in U.S. Pat. No. 6,025,134.

Methods for preparing and using nucleic acid probes and primers are described, for example, in Sambrook et al. (ed.), Molecular Cloning: A Laboratory Manual, 2^{nd} ed., vol. 1-3, Cold Spring Harbor Laboratory Press, Cold Spring Harbor, N.Y., 1989; Ausubel et al. Short Protocols in Molecular Biology, 4^{th} ed., John Wiley & Sons, Inc., 1999; and Innis et al. PCR Protocols, A Guide to Methods and Applications, Academic Press, Inc., San Diego, Calif., 1990. Amplification primer pairs can be derived from a known sequence, for example, by using computer programs intended for that purpose such as Primer (Version 0.5, © 1991, Whitehead Institute for Biomedical Research, Cambridge, Mass.). One of ordinary skill in the art will appreciate that the specificity of a particular probe or primer increases with its length. Thus, in order to obtain greater specificity, probes and primers can be selected that comprise at least 20, 25, 30, 35, 40, 45, 50 or more consecutive nucleotides of a target nucleotide sequences.

A random primer is a primer with a random sequence (see, for instance, U.S. Pat. Nos. 5,043,272 and 5,106,727). “Random” sequence in this context means that the positions of alignment and binding (annealing) of the primers to a template nucleic acid molecule are substantially indeterminate with respect to the template under conditions wherein the primers are used to initiate polymerization of a complementary nucleic acid. Methods for estimating the frequency at which an oligonucleotide of a certain sequence will appear in a nucleic acid polymer are described in Volinia et al. (Comp. App. Biosci., 5: 33-40, 1989).

The term “random primer” specifically includes a collection of individual oligonucleotides of different sequences, for instance which can be indicated by the generic formula 5′-XXXXXX-3′, wherein X represents a nucleotide residue (or modified nucleotide residue) that was added to the oligonucleotide from a mixture of a definable percentage of each dNTP. For instance, if the mixture contained 25% each of dATP, dCTP, dGTP, and dTTP, the indicated primer would contain a mixture of oligonucleotides that each have a roughly 25% average chance of having A, C, G, or T at each position. Random primers may contain modified nucleotides, such as nucleotides containing a modified base, a modified sugar moiety, and/or a modified phosphate backbone.

A sequence-specific primer, as used herein, is a primer that is designed to be complementary to a particular sequence of interest (a target sequence), or a sequence adjacent to a sequence of interest. Sequence-specific primers are designed to hybridize to, and prime replication of, a specific sequence that is to be maintained in an amplification reaction, and in many instances the specific sequence is targeted for further analysis. Sequence-specific primers are generally 5 to 60 nucleotides in length, in some instances are 15 to 30 nucleotides in length, or about 20 to 23 nucleotides in length. Sequence-specific primers may contain modified nucleotides, such as nucleotides containing a modified base, a modified sugar moiety, and/or a modified phosphate backbone.

Read: A contiguous sequence generated from a ZMW using PacBio sequencing, which includes an insert sequence and may include adapter sequence(s). A read is composed of alternating subreads and adapters.

Real-time PCR: A method for detecting and measuring products generated during each cycle of a PCR, which are proportionate to the amount of template nucleic acid prior to the start of PCR. The information obtained, such as an amplification curve, can be used to determine the presence of a target nucleic acid (such as a M. pneumoniae, C. pneumoniae, or Legionella spp. nucleic acid) and/or quantitate the initial amounts of a target nucleic acid sequence. Exemplary procedures for real-time PCR can be found in “Quantitation of DNA/RNA Using Real-Time PCR Detection” published by Perkin Elmer Applied Biosystems (1999); PCR Protocols (Academic Press, New York, 1989); and A-Z of Quantitative PCR, Bustin (ed.), International University Line, La Jolla, Calif., 2004.

In some examples, the amount of amplified target nucleic acid (for example a M. pneumoniae CARDS toxin nucleic acid molecule, a C. pneumoniae ArgR nucleic acid, a Legionella spp. SsrA nucleic acid, and/or a human RNase P nucleic acid) is detected using a labeled probe, such as a probe labeled with a fluorophore, for example a TAQMAN® probe. In this example, the increase in fluorescence emission is measured in real-time, during the course of the real-time PCR. This increase in fluorescence emission is directly related to the increase in target nucleic acid amplification. In some examples, the change in fluorescence (dRn) is calculated using the equation dRn=Rn⁺−Rn⁻, with Rn⁺ being the fluorescence emission of the product at each time point and Rn⁻ being the fluorescence emission of the baseline. The dRn values are plotted against cycle number, resulting in amplification plots for each sample. The threshold value (Cₜ) is the PCR cycle number at which the fluorescence emission (dRn) exceeds a chosen threshold, which is typically 10 times the standard deviation of the baseline (this threshold level can, however, be changed if desired).

The threshold cycle is when the system begins to detect the increase in the signal associated with an exponential growth of PCR product during the log-linear phase. This phase provides information about the reaction. The slope of the log-linear phase is a reflection of the amplification efficiency. The efficiency of the reaction can be calculated by the following equation: E=10^{(-1/slope)}. The efficiency of the PCR should be 90-100% meaning doubling of the amplicon at each cycle. This corresponds to a slope of −3.1 to −3.6 in the Cₜ vs. log-template amount standard curve. In order to obtain accurate and reproducible results, reactions should have efficiency as close to 100% as possible (meaning a two-fold increase of amplicon at each cycle).

Reverse Transcriptase: A template-directed DNA polymerase that generally uses RNA but can use DNA as its template.

Reversibly binding to a target nucleic acid molecule: Temporary binding that exists in a reversible equilibrium. For example, includes transient pairing of a nucleotide to its complement at the active site of a polymerase, wherein the nucleotide does not undergo a chemical reaction (such as hydrolysis or covalent bond formation) that covalently incorporates the nucleotide into the nucleic acid molecule being formed by the polymerase.

RNA polymerase: An enzyme that catalyzes the polymerization of ribonucleotide precursors that are complementary to the DNA template.

Sample: A portion, piece, or segment that is representative of a whole. This term encompasses any material, including for instance samples obtained from an animal, a plant, or the environment.

Specifically contemplated samples include sources of one or more nucleic acid molecules (e.g., DNA or RNA), such as material from an animal or plant source.

Samples include biological samples such as those derived from a human or other animal source (for example, blood, stool, sera, urine, saliva, tears, tissue biopsy samples, surgical specimens, histology tissue samples, autopsy material, cellular smears, embryonic or fetal cells, amniocentesis or chorionic villus samples, etc.); bacterial or viral or other microbial preparations; cell cultures; forensic samples; agricultural products; waste or drinking water; milk or other processed foodstuff; air; and so forth. Samples suitable for disclosed methods include nucleic acid molecules (e.g., DNA or RNA).

A sample can contain multiple cells, a single cell, no intact cells at all, or can be prepared from cells, such as from a single cell, for instance a nucleus. Samples of limited quantity are contemplated, such as biopsies (such as tumor biopsies), forensic samples, archived DNA or tissue samples, and embryo biopsies and other embryo and pre-embryo samples (such as cells from an in vitro fertilization). Samples containing a small number of cells, or a single cell, can be acquired by any one of a number of methods, such as fine needle aspiration, micro-dissection, biopsy, tissue scrapes, forensic swabs, or laser capture micro-dissection. Samples can also be diluted to a level where they contain as few as 100 cells, ten cells, or even as few as one cell in a sample, and used e.g., for subsequent analysis.

Samples may also be a biological or non-biological material that contains trace amounts of “contaminating” biological materials. For example, methods described herein are specifically contemplated for use in detecting the presence of bacteria or viruses in a sample such as food, water, drugs, an otherwise inert powder, a package, or other item. Samples include any item that may contain, or be contaminated, with a microbe or infectious agent, particularly a biological agent that could cause disease and/or be used for bioterrorism. Samples also include food or water, or other materials that may contain or be contaminated with a microbe, such as a disease- or illness-causing microbe, and drug preparations, such as those that are prepared using recombinant DNA technology.

An “environmental sample” includes a sample obtained from inanimate objects or reservoirs within an indoor or outdoor environment. Environmental samples include, but are not limited to: soil, water, dust, and air samples; bulk samples, including building materials, furniture, and landfill contents; and other reservoir samples, such as animal refuse, harvested grains, and foodstuffs.

A “biological sample” is a sample obtained from a plant or animal subject. As used herein, biological samples include all samples useful for detection of viral infection in subjects, including, but not limited to: cells, tissues, and bodily fluids, such as blood; derivatives and fractions of blood (such as serum); extracted galls; biopsied or surgically removed tissue, including tissues that are, for example, unfixed, frozen, fixed in formalin and/or embedded in paraffin; tears; milk; skin scrapes; surface washings; urine; sputum; cerebrospinal fluid; prostate fluid; pus; bone marrow aspirates; BAL; saliva; cervical swabs; vaginal swabs; and oropharyngeal wash.

A “forensic sample” is a sample that may be used for the application of science or technology in the investigation and establishment of facts or evidence, for instance for use in a court of law. A forensic sample is often a sample taken from a non-biological source that is used to extract biological material that may be used for the isolation and analysis of DNA or RNA. One example of a forensic sample is a piece of carpet that contains drops of blood. The blood may be extracted from the carpet, such as by collection with a swab, and DNA or RNA can subsequently be isolated using standard techniques. Examples of biological materials that may be used for forensic testing include, but are not limited to, blood, saliva, semen, urine or feces, hair, skin, bone, and other body tissues.

Sequence Identity: The similarity between two nucleic acid sequences, or two amino acid sequences, is expressed in terms of the similarity between the sequences, otherwise referred to as sequence identity. Sequence identity is frequently measured in terms of percentage identity (or similarity or homology); the higher the percentage, the more similar the two sequences are.

Methods of alignment of sequences for comparison are well known in the art. Various programs and alignment algorithms are described in: Smith and Waterman (Adv. Appl. Math., 2:482, 1981); Needleman and Wunsch (J. Mol. Biol., 48:443, 1970); Pearson and Lipman (Proc. Natl. Acad. Sci., 85:2444, 1988); Higgins and Sharp (Gene, 73:237-44, 1988); Higgins and Sharp (CABIOS, 5:151-53, 1989); Corpet et al. (Nuc. Acids Res., 16:10881-90, 1988); Huang et al. (Comp. Appls. Biosci., 8:155-65, 1992); and Pearson et al. (Meth. Mol. Biol., 24:307-31, 1994). Altschul et al. (Nature Genet., 6:119-29, 1994) presents a detailed consideration of sequence alignment methods and homology calculations.

The alignment tools ALIGN (Myers and Miller, CABIOS 4:11-17, 1989) or LFASTA (Pearson and Lipman, 1988) may be used to perform sequence comparisons (Internet Program © 1996, W. R. Pearson and the University of Virginia, “fasta20u63” version 2.0u63, release date December 1996). ALIGN compares entire sequences against one another, while LFASTA compares regions of local similarity. These alignment tools and their respective tutorials are available on the Internet at the NCSA website. Alternatively, for comparisons of amino acid sequences of greater than about 30 amino acids, the “Blast 2 sequences” function can be employed using the default BLOSUM62 matrix set to default parameters, (gap existence cost of 11, and a per residue gap cost of 1). The BLAST sequence comparison system is available, for instance, from the NCBI web site; see also Altschul et al., J. Mol. Biol., 215:403-10, 1990; Gish and States, Nature Genet., 3:266-72, 1993; Madden et al., Meth. Enzymol., 266:131-141, 1996; Altschul et al., Nucleic Acids Res., 25:3389-3402, 1997; and Zhang and Madden, Genome Res., 7:649-56, 1997.

Similar homology concepts apply for nucleic acids and for protein. An alternative indication that two nucleic acid molecules are closely related is that the two molecules hybridize to each other under stringent conditions. Nucleic acid sequences that do not show a high degree of identity may nevertheless encode similar amino acid sequences, due to the degeneracy of the genetic code. It is understood that changes in nucleic acid sequence can be made using this degeneracy to produce multiple nucleic acid sequences that each encode substantially the same protein.

Sequence of signals: The sequential series of emission signals, including fri instance electromagnetic signals such as light or spectral signals, which are emitted upon specific binding of chemical moieties (such as a nucleotide analog) with complementary nucleotides in the target nucleic acid molecule, which indicates pairing of the chemical moiety with its complementary nucleotide. In a particular example, the sequence of signals is a series of acceptor fluorophore emission signals, wherein each unique signal is associated with a particular chemical moiety.

Sequencing (a nucleic acid molecule): Any of several methods and technologies that are used to determine the order of the nucleotide bases (adenine, guanine, cytosine, and thymine or uracil) in a molecule of DNA (or RNA).

Signal: A detectable change or impulse in a physical property that provides information. In the context of the disclosed methods, examples include electromagnetic signals such as light, for example light of a particular quantity or wavelength. In certain examples the signal is the disappearance of a physical event, such as quenching of light.

Strand displacement activity: The ability of a polymerase to displace a hybridized downstream (non-template) DNA strand encountered during synthesis. Displacement of a DNA strand makes the displaced strand available as template for primer hybridization and DNA replication. Examples of DNA polymerases with strand displacement activity include, but are not limited to, Phi29 DNA polymerase, Bst DNA polymerase, Vent_{R}™ and Deep Vent_{R}™ DNA polymerases, 9° Nₘ DNA polymerase, Klenow fragment of DNA polymerase I, PhiPRD1 DNA polymerase, phage M2 DNA polymerase, T4 DNA polymerase, and T5 DNA polymerase.

In contrast to polymerases with strand displacement activity, some polymerases (such as Taq DNA polymerase) degrade downstream hybridized DNA encountered during synthesis via a 5′-3′ exonuclease activity.

Subread: Sequence generated in a PacBio sequencing system by splitting the raw sequence (read) from a ZMW at the adapter sequences. This is the post-sequencing version of the “insert DNA” template used in sample preparation.

Template nucleic acid: A nucleic acid strand that is the substrate for synthesis of a complementary nucleic acid, such as by the annealing of a primer and extension by a DNA polymerase, or by reverse transcribing DNA from an RNA template.

Under conditions sufficient for: A phrase that is used to describe any environment that permits the desired activity.

An example includes contacting a probe with a sample under conditions sufficient to allow sequencing of a target nucleic acid molecule in the sample, for example to determine whether the target nucleic acid molecule is present in the sample, such as a target nucleic acid molecule containing one or more mutations.

Zero-mode waveguide (ZMW): A nanophotonic confinement structure that consists of a circular hole in an aluminum cladding film deposited on a clear silica substrate (Korlach et al., Proc Natl Acad Sci 105:1176-1181, 2008). The ZMW holes are ˜70 nm in diameter and ˜100 nm in depth. Due to the behavior of light when it travels through a small aperture (the bottom of the ZMQ), the optical field decays exponentially inside the chamber (Foquet et al., J. Appl. Phys. 103: 034301-1-034301-9, 2008; available on-line at dx.doi.org/10.1063/1.2831366). The observation volume within an illuminated ZMW is ˜20 zeptoliters (20×10⁻²¹ liters). A sequencing ZMW is one that is expected to be able to produce a sequence if it is populated with a polymerase.

Unless otherwise explained, all technical and scientific terms used herein have the same meaning as commonly understood by one of ordinary skill in the art to which this invention belongs. The singular terms “a,” “an,” and “the” may include the plural equivalent. Similarly, the word “or” is intended to include “and” unless the context clearly indicates otherwise. Hence “comprising A or B” means including A, or B, or A and B. It is further to be understood that all base sizes or amino acid sizes, and all molecular weight or molecular mass values, given for nucleic acids or polypeptides are approximate. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including explanations of terms, will control. In addition, the materials, methods, and examples are illustrative only and not intended to be limiting.

### III. Overview of Several Embodiments

Provided herein in a first embodiment is a method of sequencing a pool of at least two amplicons having different lengths, the method involving mixing an amount of a first amplicon with an amount of a second amplicon, wherein the amounts of the first and second amplicons are selected so there is a molar excess of the longer of the two amplicons in the resultant pooled amplicons; and subjecting the pooled amplicons to a nucleic acid sequencing reaction. Optionally, the molar excess is at least a linear molar excess based on the relative length of the amplicons, such that an amplicon that is twice as long will be present twice as often in the resultant pool.

Pools of amplicons in the described methods can include any number of different amplicons. Thus, in some embodiments, at least 10 amplicons are pooled, or at least 50 amplicons are pooled, or at least 100 amplicons are pooled, or even over (that is, more than) 100 amplicons are pooled.

Without intending to be limited thereby, one particularly contemplated embodiments is use of the sequence refinements described herein in the context of single-molecule real-time (SMRT) sequencing, such as PacBio sequencing. Thus, provide embodiments include methods of sequencing a pool of at least two amplicons having different lengths, the method involving mixing an amount of a first amplicon with an amount of a second amplicon, wherein the amounts of the first and second amplicons are selected so there is a molar excess of the longer of the two amplicons in the resultant pooled amplicons; and subjecting the pooled amplicons to a single-molecule real-time (SMRT) nucleic acid sequencing reaction.

The sequencing methods provided herein can be used in genome assembly, for instance, wherein one or more of the amplicons in the sequencing pool bridges at least one known or suspected gap in a genome assembly. Specifically contemplated are methods wherein at least one gap bridged by an amplicon being sequenced is at least 50 bp, at least 100 bp, at least 200 bp, at least 300 bp, at least 400 bp, at least 500 bp, at least 600 bp, at least 700 bp, at least 800 bp, at least 900 bp, at least 1 Kb, at least 1.2 Kb, at least 1.3 Kb, at least 1.4 Kb, at least 1.5 Kb, at least 1.6 Kb, at least 1.7 Kb, at least 1.8 Kb, or at least 1.9 Kb in length. Also contemplated are methods wherein at least one gap is at least 2 Kb in length, and methods wherein at least one gap is more than 2 Kb in length.

By way of example, representative embodiments of sequencing methods provided herein that are used in genome assembly involve subjecting the amplicon (or pool of amplicons) to serial sequencing to produce a series of subreads of the same amplicon template; selecting a subset of the subreads based on the accuracy of the sequence of a portion of the amplicon; and using the sequences of the subset of subreads to assemble a consensus sequence for the amplicon.

Another provided embodiment is an improved method for single-molecule real-time (SMRT) sequencing a pool of amplicons having different lengths, wherein the improvement comprises adjusting the amount of at least two of the amplicons included in the pool using the following formula: Volume=[PCR size (Kb)]²×[10 ng/PCR concentration (ng/μl)].

Also provided herein is a method for gap-filling sequencing of at least one amplicon, which method involves subjecting the amplicon to serial sequencing to produce a series of subreads of the same amplicon template; selecting a subset of the subreads based on the accuracy of the sequence of a portion of the amplicon; and using the sequences of the subset of subreads to assemble a consensus sequence for the amplicon. Optionally, the serial sequencing comprises single-molecule real-time (SMRT) sequencing.

By way of example, the portion of the amplicon is at least 30 nucleotides, at least 50 nucleotides, at least 70 nucleotides, or at least 100 nucleotides in length. Longer portions are also contemplated, though the overall length will be influenced by amount of template length that is known and the size of the gap that is being filled. The gap can be of any length, for instance at least 50 bp, at least 100 bp, at least 200 bp, at least 300 bp, at least 400 bp, at least 500 bp, at least 600 bp, at least 700 bp, at least 800 bp, at least 900 bp, at least 1 Kb, at least 1.2 Kb, at least 1.3 Kb, at least 1.4 Kb, at least 1.5 Kb, at least 1.6 Kb, at least 1.7 Kb, at least 1.8 Kb, or at least 1.9 Kb in length. Also contemplated are methods wherein at least one gap is at least 2 Kb in length, and methods wherein at least one gap is more than 2 Kb in length.

In examples of this method, the portion of the amplicon is a unique sequence in the context of the sequencing reaction.

By way of example, in some embodiments the subset of subreads comprises at least 50, at least 100, at least 150, or at least 200 subreads of the same amplicon template. In other examples of the method, for instance where the gap to be filled is relatively long, the subset of subreads is larger and comprises for instance at least 300 subreads or more of the same amplicon template.

### IV. Single Molecule Real Time (SMRT) Sequencing

Single molecule real time sequencing (SMRT) is a parallelized single molecule DNA sequencing by synthesis technology developed by Pacific Biosciences. Single molecule real time sequencing utilizes the zero-mode waveguide (ZMW) (Levene et al., Science 299:682-685, 2003). A single DNA polymerase enzyme is affixed at the bottom of a ZMW with a single molecule of DNA as a template. The ZMW is a structure that creates an illuminated observation volume that is small enough to observe only a single nucleotide of DNA being incorporated by DNA polymerase. Each of the four DNA bases is attached to one of four different fluorescent dyes. When a nucleotide is incorporated by the DNA polymerase, the fluorescent tag is cleaved off and diffuses out of the observation area of the ZMW where its fluorescence is no longer observable. A detector detects the fluorescent signal of the nucleotide incorporation, and the base call (identification of the incorporated nucleotide) is made according to the corresponding fluorescence of the dye. Sequence data generated from single molecule real time sequencing was first published in January 2009 (Eid et al., Science 323:133-138, 2009). SMRT sequencing is carried out on a chip that contains many ZMWs. Additional information about SMRT sequencing and the construction and loading of ZMWs can be found, for instance, in US Published Application No. 2010-0009872, which is incorporated herein by reference in its entirety.

SMRT sequencing can be used, for instance, for de novo sequencing. Read lengths from the single molecule real time sequencing are comparable to or greater than that from the Sanger sequencing method based on dideoxynucleotide chain termination. The longer read length allows de novo genome sequencing and easier genome assemblies (Eid et al., Science 323:133-138, 2009). See also Rasko et al. (N Engl. J Med. 365:709-717, 2011) and Chin et al. (N Engl. J Med. 364:33-42, 2011), describing use of SMRT sequencing for de novo genome sequence analysis of the E. coli outbreak in Germany in 2011 and in the cholera outbreak in Haiti in 2010, respectively. SMRT sequencing has also been used in hybrid assemblies for de novo genomes to combine short-read sequence data with long-read sequence data.

SMRT sequencing is also employed in re-sequencing methods. A DNA molecule can be re-sequenced independently by creating the circular DNA template (using adaptors—hairpin loops ligated to both ends of the double stranded DNA template) and utilizing a strand displacing enzyme that separates the newly synthesized DNA strand from the template. This circular consensus sequencing (CCS) approach has been used with SMRT sequencing (Smith et al., Nature 2012; doi:10.1038/nature11016). When the adaptor sequences are removed from raw sequence data (the read, which contains alternating subreads and adaptors)), the read is split into multiple subreads.

### IV. Sequencing Improvements

Described herein are methods that overcome, for instance, the loading bias against larger PCR products in the PacBio technology. This is accomplished by adjusting the amount of amplicons mixed to form a sequencing pool so that DNA molecules having longer sequences are more highly represented than those with shorter sequences. The volume of each amplicon in the pool varies by the square of its length but the molarity of each amplicon in the pool has a linear relationship to the length of the amplicon—thus, an amplicon of ˜2 Kb would be present in the pool approximately two times more often than an amplicon of ˜1 Kb, and one of ˜3 Kb would present approximate three times more often.

The resultant reduced loading bias method provides an efficient system for pooling PCR products (including more than one hundred different PCR products) into one sequencing library and generating good sequencing coverage using PacBio SMRT sequencing, even when PCR products in the pool are of various sizes. Such pooled sequencing is a more efficient and economical method to close gaps in draft genomes since larger gaps can be closed with the PacBio technology.

Though exemplified using PacBio SMRT sequencing, the improvements described herein are useful with any sequencing platform used for sequencing pools of different sized nucleic acids and which exhibits a small molecule bias. It is particularly beneficial with platforms that generate sequence read length of 2 kb or longer, as elimination of the small molecule bias from such systems enables filling of long gaps for instance for genome assembly.

PacBio SMRT sequencing currently involves extensive computer analysis of raw sequence reads, one aspect of which is removal of adaptor sequence in order to yield subreads that contain template sequence and possibly some portion of the adaptor sequence. The process as carried out by software provided, for instance, with the PacBio RS device, removes adaptors in between the target sequences and filters out resulting subreads of less than 50 bp and those with quality determined to be less than 75% (calculated by PacBio's algorithms).

Provided herein is a refinement to this process, in which unique-sequence primer tags are used along with an additional length (for instance, ˜150 bp) of unique template sequence adjacent to the primer to BLAST against the subreads. By choosing only those subreads (for instance ˜200 subreads, or ˜300 subreads particularly for amplicons >2.5 kb) that have the highest identity to these unique sequences and to create a consensus sequence for the amplicon, the quality of resultant sequence data for each amplicon is significantly improved. For smaller gaps where the missing sequences were resolved by both Sanger and PacBio technologies, using this primer-plus-150 bp-unique template sequence screening system 91% of the PacBio consensus sequences matched the Sanger sequences with a 98% identity or better. Similar results have been found for identity to known sequence with larger PacBio PCR amplicons across regions of known sequence.

This template unique sequence screening to select the “better” subreads can be used in conjunction with the modification of amplicon pooling components, though that is not essential. Each of these improvements can be used on its own, though embodiments provided herein exemplify the two being used together as well.

The methods described in this application can be applied to any situation where amplicons of different sizes compete for a binding partner. For example, in the case of nucleotide sequencing using the PacBio platform, amplicons of different size compete for the polymerase immobilized at the bottom of each zero-mode waveguide (ZMW) chamber. Amplicons of smaller size have competitive binding advantage to get into the ZMW chambers compared to those of larger size, which results in a bias in the binding complex distribution. Because of this bias, a major fraction of the subreads are generated from smaller size amplicons in PacBio sequencing platform.

To attenuate this bias, described herein is development of a formula that enables increasing the molar amount of amplicons of larger size in the sequencing template mixture to generate a better distribution of subreads for amplicons of different sizes. The specific formula provided herein can be modified or further optimized to reduce binding bias caused by amplicons of different sizes in a wide molar concentration range for different purposes, including, but not limited to, closing sequencing gaps.

The following examples are provided to illustrate certain particular features and/or embodiments. These examples should not be construed to limit the invention to the particular features or embodiments described.

### EXAMPLES

Improving the quality of genomes produced from advanced sequencing technologies requires an efficient and economical means to close gaps and resequence some regions in the genomes. Sequencing pooled PCR products with PacBio (McCarthy, Chem Biol. 17:675-676, 2010; Schadt et al., Hum Mol Genet. 19(R2):R227-240, 2010) provides a significantly less expensive means for the need. We have developed and describe herein techniques for overcoming the loading bias inherent in the PacBio sequencing process; this improvement can be included in genome improvement pipelines that employ pooled PCR sequencing strategies. Compared to Sanger technology (Pons, J Assoc Off Anal Chem., 58:746-753, 1975; Tabor & Richardson, Proc Natl Acad Sci USA 84:4767-4771, 1987), the herein described-approach is not only cost-effective but also can close gaps greater than 2.5 Kb in a single round of reactions. It can also sequence through high GC regions (e.g., as described in Ji et al., Nuc Acids Res. 24:2835-2840, 1996) and difficult secondary structures such as hairpin loops.

Second-generation sequencing technologies produce more and more draft genomes at an ever faster speed and lower cost. However, finished high quality genomes are still preferably used by researchers (Chain et al., Science 326:236-237, 2009). Closing gaps in a draft genome is necessary to improve the quality of the genome. Picking primers at gap regions for PCR and assembling the resulting PCR sequences into the genome can reduce numbers of both contigs and scaffolds. Since the advancement of much less expensive sequencing technologies, Sanger sequencing (Sanger et al., Nature 265:687-695, 1977; Sanger et al., Proc Natl Acad Sci USA, 70, 1209-1213, 1973) of individual PCR products spanning targeted regions becomes a more expensive method compared to the cost of the draft itself. Pooling dozens of PCR products of various sizes and sequencing them as one library with single molecular sequencing technology from PacBio is a much more economical option (McCarthy, Chem Biol. 17:675-676, 2010; Schadt et al., Hum Mol Genet. 19(R2):R227-240, 2010).

However, there is a loading bias against large DNA fragments in the PacBio sequencing process. The PacBio technique uses single molecule sequencing done in wells on a chip, which is called a Single Molecule Real Time (SMRT) cell. Smaller PCR products will load into the PacBio wells with a much greater efficiency than larger PCR products. When PCR products ranging from 500 bp to 5 Kb are pooled and sequenced together using PacBio, the smaller products have a substantially higher coverage than the larger products resulting in poor quality or incomplete sequences for the larger PCR products.

To address this problem, the molar ratio of the PCR products was adjusted when pooling them together based on the PCR size and concentration. This resulted in a much closer distribution of coverage for the different sizes of PCR products. A finished genome was used to normalize this process, and 18 PCR primer pairs with amplification sizes ranging from 500 bp to 5 Kb were chose. The PCRs were performed using commercial kits: FailSafe™ PCR System (Epicenter) for genomes with mid-range GC content (40-60%) and GC-Rich PCR System (Roche) for genomes with GC content higher than 60%. PCR products were cleaned individually (ZR 96 DNA Clean and Concentrator, Zymo Research) and pooled PCRs were purified again (Agencourt AMPure XP, Beckman Coulter). The results were pooled into three groups with three different approaches:

Group one (control): equal DNA mass was loaded for every PCR product;

Group two: PCR products were pooled at the equal molar amount for each; and

Group three: the molar mass ratio was adjusted based on the size of the PCR, increasing the molar amount with the PCR size.

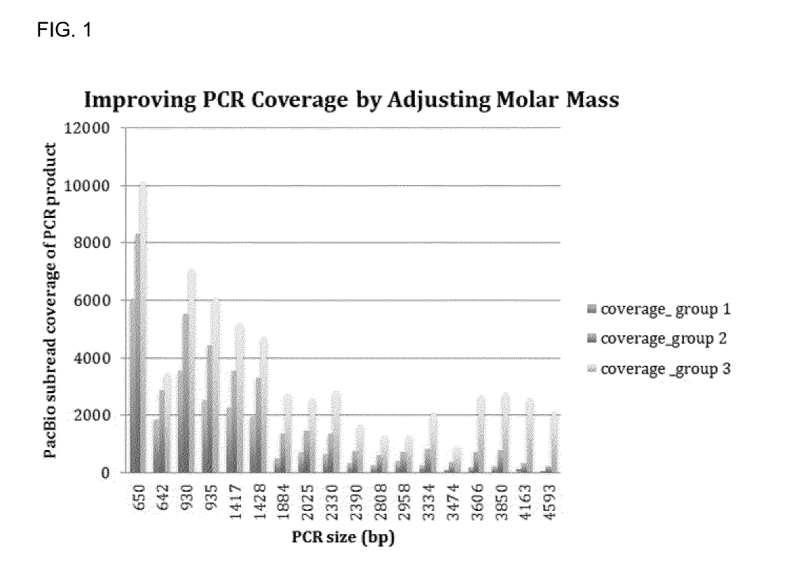

The results are shown in FIG. 1. The control group resulted in much higher coverage for the smaller PCR products while the longer PCR products were barely covered (shown in the first bar in each set of bars in FIG. 1). The second group had an improvement in coverage for the larger products, but still less than the coverage for the smaller products (shown in the second bar in each set of bars in FIG. 1). The third group shows dramatic improvement in the coverage for the larger products (shown in the third bar in each set of bars in FIG. 1).

The formula below was used to make the molar amount adjustment to obtain a relative molar excess of longer amplicons based on size and concentration, and to calculate the volume needed for each PCR and for robotic pooling:

Volume=[PCR size (Kb)]²×[10 ng/PCR concentration (ng/μl)]

This formula permits adjustment of the molar amount of amplicons of different sizes and concentrations, thus attenuating the sequencing bias inherent in prior PacBio sequencing methods caused by amplicons of different sizes. Using the above formula, one increases the molar amount of amplicons of larger size in a sequencing template mixture (pool) to generate a better distribution of subreads for amplicons of different sizes.

Amplicons' size and concentration can be collected from upstream measurements (gel electrophoresis or commercial instruments like QIAxcel system from Qiagen, NanoDrop, or Caliper LCGX, etc.). After one gets the volume calculated from the formula and size and concentration, the molar amount of each amplicon can be calculated by:

Molar amount (mol)=concentration×volume/molecular weight

where molecular weight (MW) is size dependent, as can be calculated below:

MW of dsDNA (g/mol)=# nucleotides×607.4+157.9

The molecular weight of each nucleotide in a DNA molecule (A, T, C, G) are different, but the difference is too small to affect the molar amounts.

The formula can be modified according to the size and concentration reading from different upstream source or according to the molar amount requirement for downstream analysis.

Based on this calculation, the volume of each amplicon added to the resultant pool varies directly with the square of its length assuming that the starting concentration of each amplicon is equal. The volume of each amplicon in the pool varies by the square of its length but the molarity of each amplicon in the pool is a linear relationship to the length of the amplicon. During the volume calculation, the square of length in the numerator (above) is canceled out by the length in the denominator, which leaves only a linear relationship between molar amount and amplicon size.

We have combined over 200 PCRs (amplicons) into one pool and the above-described adjustment process produced good sequencing coverage for the products. Since one SMRT cell can produce 0.5 gigabases of data (after filtering to remove adapters), the process described in this example provides an efficient method of pooling 500-1000 PCR products into one sequencing library depending on the sizes of the PCR products. By decreasing the loading bias against larger PCR products that has thus far been inherent in the PacBio technology, we have developed a much more efficient and economical method to close gaps in draft genomes, since larger gaps can be closed with the PacBio technology (long reads) than with prior sequencing technologies.

The above-described gap closure method has been applied to sixteen bacterial genome projects in our genome improvement pipeline. Primers for 362 regions in these sixteen projects were selected and the resulting products sequenced with both Sanger (Pons, J Assoc Off Anal Chem., 58:746-753, 1975; Tabor & Richardson, Proc Natl Acad Sci USA 84:4767-4771, 1987) and PacBio technologies (McCarthy, Chem Biol. 17:675-676, 2010; Schadt et al., Hum Mol Genet. 19(R2):R227-240, 2010). The gap sizes ranged from 500 bp to 5 Kb. While the majority of gaps less than 2.5 Kb were closed with both Sanger (64%) and PacBio (73%) technologies, none of the gaps larger than 2.5 Kb were closed with a single round of Sanger technology. PacBio sequencing of the PCR products using the loading bias correction described above closed almost 90% of these larger gaps.

This method also allows the closure of gaps due to small hairpin structures (typically with higher GC content) where other sequencing technologies usually fail, since PacBio can successfully sequence through these regions. Hard stops are regions with strong secondary structures in a DNA template may form hairpin structures that prevent DNA polymerase from passing through, which makes it difficult to sequence these regions (see Table 1.)

Because one of our goals is to reduce costs, we pool over one hundred PCR products in a single PacBio SMRT cell for sequencing. To successfully assemble the PacBio subreads (a sub-portion of a read resulted from screening and removing of sequencing adapters that were in the middle of a read) into an accurate consensus for a single PCR product, we pull out subreads from the pool of sequenced subreads that belong only to that PCR product. We developed computational scripts to interact with our local database to identify the primer sequences and an additional 150-nucleotide unique sequence next to the primers from the draft assembly to fish out the subreads (using BLAST; Altschul et al., Nuc. Acids Res. 25:3389-3402, 1997) that belong to a particular PCR product and therefore, a particular gap. This is especially necessary for repeat gaps so that if there are slight differences in the repeats, they can be resolved correctly.

Since the error rate of PacBio sequencing is typically reported to be about 15%, we developed a further refinement to increase the accuracy of the consensus sequence obtained. By choosing 200 subreads with the highest sequence match to the primer-plus-150 nt-unique sequences, we were able to dramatically improve the quality of the PCR product consensus sequences after assembling the selected subreads for an individual PCR product using ALLORA, the long read assembler for de novo assembly from PacBio (Pacific Biosciences, Menlo Park, Calif.). For the smaller gaps where the missing sequences were resolved by both Sanger and PacBio technologies, 91% of the PacBio consensus sequences matched the Sanger sequences with a 98% identity or better. To try to maintain a roughly equivalent accuracy rate, for the larger PCR products we increased the number of selected subreads to 300. We did not see a significant difference in the results based on the GC content of the genomes. For genomes with mid-range GC content (40-60%), 78% of 51 PCRs closed the gap. For genomes with high GC content (>60%), 86% of 311 PCRs closed gaps.

As illustrated in Table 1, PacBio with the modifications as described herein (which include molar amount adjustment and generating consensus sequence for each amplicon) closes larger gaps and hard stops in a single round of PCR. 362 PCR products (each covers a different gap) were sequenced with both Sanger and PacBio technologies. While the majority of gaps less than 2.5 Kb were closed with both Sanger (64%) and PacBio (73%) technologies, none of the gaps larger than 2.5 Kb were closed with a single round of Sanger technology. Three hard stop gaps that could not be closed using Sanger sequencing were all closed using PacBio as described herein.

TABLE 1

| PCR | # PCR | % closed by Sanger | % closed by PacBio |
|---|---|---|---|
| <2.5 kb | 246 | 64 | 73 |
| >2.5 kb | 113 | 0 | 88 |
| hairpin structure | 3 | 0 | 100 |

This disclosure provides methods of enhancing high throughput sequencing techniques, including methods that reduce template-length-based loading bias. It will be apparent that the precise details of the methods described may be varied or modified without departing from the spirit of the described invention. We claim all such modifications and variations that fall within the scope and spirit of the claims below.

1. A method of sequencing a pool of at least two amplicons having different lengths, the method comprising:
mixing an amount of a first amplicon with an amount of a second amplicon, wherein the amounts of the first and second amplicons are selected so there is a molar excess of the longer of the two amplicons in the resultant pooled amplicons; and
subjecting the pooled amplicons to a nucleic acid sequencing reaction.

2. The method of claim 1, wherein molar excess is at least a linear molar excess based on the relative length of the amplicons.

3. The method of claim 1, wherein at least 10 amplicons are pooled.

4. The method of claim 1, wherein at least 50 amplicons are pooled.

5. The method of claim 1, wherein at least 100 amplicons are pooled.

6. The method of claim 1, wherein over 100 amplicons are pooled.

7. The method of claim 1, wherein the sequencing reaction comprises single-molecule real-time (SMRT) sequencing.

8. The method of claim 1, wherein the amplicons bridge known or suspected gaps in a genome assembly.

9. The method of claim 8, wherein at least one gap is at least 50 bp, at least 100 bp, at least 200 bp, at least 300 bp, at least 400 bp, at least 500 bp, at least 600 bp, at least 700 bp, at least 800 bp, at least 900 bp, at least 1 Kb, at least 1.2 Kb, at least 1.3 Kb, at least 1.4 Kb, at least 1.5 Kb, at least 1.6 Kb, at least 1.7 Kb, at least 1.8 Kb, or at least 1.9 Kb in length.

10. The method of claim 8, wherein at least one gap is at least 2 Kb in length.

11. The method of claim 8, wherein at least one gap is more than 2 Kb in length.

12. The method of claim 8, wherein the sequencing reaction comprises for each amplicon:
subjecting the amplicon to serial sequencing to produce a series of subreads of the same amplicon template;
selecting a subset of the subreads based on the accuracy of the sequence of a portion of the amplicon; and
using the sequences of the subset of subreads to assemble a consensus sequence for the amplicon.

13. An improved method for single-molecule real-time (SMRT) sequencing a pool of amplicons having different lengths, wherein the improvement comprises adjusting the amount of at least two of the amplicons included in the pool using the following formula:
Volume=[PCR size (Kb)]²×[10 ng/PCR concentration (ng/μl)].

14. A method for gap-filling sequencing of at least one amplicon, comprising:
subjecting the amplicon to serial sequencing to produce a series of subreads of the same amplicon template;
selecting a subset of the subreads based on the accuracy of the sequence of a portion of the amplicon; and
using the sequences of the subset of subreads to assemble a consensus sequence for the amplicon.

15. The method of claim 14, wherein the portion of the amplicon is at least 100 nucleotides in length.

16. The method of claim 14, wherein the portion of the amplicon is a unique sequence.

17. The method of claim 14, wherein the subset of subreads comprises at least 200 subreads of the same amplicon template.

18. The method of claim 16, wherein the subset of subreads comprises at least 300 subreads of the same amplicon template.

19. The method of claim 14, wherein the gap to be filled is at least 2000 base pairs in length.

20. The method of claim 18, wherein the serial sequencing comprises single-molecule real-time (SMRT) sequencing.
